(19) United States
(12) Patent Application Publication
(10) Pub. No.: US 2016/0286906 A1
(43) Pub. Date: Oct. 6, 2016

(54) METHOD AND SYSTEM FOR MEASURING 3-DIMENSIONAL OBJECTS

(71) Applicant: EDGIMAGO 2012 LTD, Karmei Yosef (IL)

(72) Inventors: Noam MALAL, Tel Aviv (IL); Omer KOREN, Givatayim (IL)

(73) Assignee: EDGIMAGO 2012 LTD., Karmei Yosef (IL)

(21) Appl. No.: 15/035,317

(22) PCT Filed: Nov. 6, 2014
(86) PCT No.: PCT/IL2014/050970

§ 371 Date: May 9, 2016

Related U.S. Application Data
(60) Provisional application No. 61/902,197, filed on Nov. 9, 2013.

(51) Int. Cl. A43D 1/02; G06T 7/00; G06T 11/60; A61B 5/107; A43B 17/00; A43D 1/06; A41H 1/00; H04N 13/02; G06K 9/62

(52) U.S. Cl. CPC A43D 1/025; H04N 13/0203; G06T 7/0022; G06T 11/60; G06K 9/6202; A43B 17/00; A43D 1/06; A41H 1/00; A61B 5/1077; A61B 5/1079; G06T 2207/10012

(57) ABSTRACT

A method for obtaining size and shape data of a subject comprising positioning a substantially two dimensional reference object on a plane near to the subject; providing a digital camera comprising a display screen, a digital imaging chip, a processor, a memory and a transmitter; imaging the object and subject on the display screen together with a framework corresponding to a projection of the reference object from a desired angle, and tilting the screen together with a framework corresponding to an outline of the reference object to align the outline with the perimeter of the reference object on the screen.

### BACKGROUND

The present invention is directed to methods and systems for modeling three dimensional objects, such as a part of the body, thereby enabling on-line purchasing of appropriate clothing and shoes, for example.

In the not too distant past, many goods purchased by customers living in central regions of the United States were purchased from catalogs. Nowadays, the Internet is widely used for purchasing a wide range of goods, in particular books, since online retailers can offer far larger ranges than a traditional store can carry, and one-off articles, such as historical artifacts and collector's items, since the Internet enables collectors and vendors to finds each other. In addition to books and collectables, consumers tend to purchase airplane tickets and tickets for shows over the Internet since the Internet enables the customer to work at his or her own pace and the product is well defined. Except for very complicated multi-stop journeys, there is little or no advantage in working with a personal travel agent and purchasing over the Internet is convenient and generally cheaper. Holiday packages, hotel rooms and self-catering flats are also booked over the Internet. Here there is little standardization between offerings, but the Internet provides as much if not more information as a printed catalog and the consumer can check that amenities of interest are provided at an appropriate standard. The consumer relies on market forces and perhaps on Ministry of Tourism or similar to ensure that the description and the product are similar and is able to read reviews from other travelers. Furthermore, the consumer is able to post his or her own review.

Increasingly, food, and other goods are purchased over the Internet. Clothing in general and shoes in particular are, however, still largely purchased from traditional stores. Such clothes may not fit properly and one wishes to try them on before purchase. Merely knowing what size shoes one generally takes is an indication of the size of shoe that might fit, but in practice, for a particular model, one often finds a larger or smaller size more appropriate, or that the model is uncomfortable and then one tries a different design. These considerations create a psychological barrier that results in clothing in general and shoes in particular not being purchased over the Internet, or, when purchased, having a high rate of return which increases transportation, storage and service costs, which are generally passed onto the customer. In principle, however, the Internet should provide a larger range of styles and cheaper unit cost for shoes and other clothing, than traditional retail outlets.

It would be useful if shoes and clothing could be purchased over the Internet with a greater degree of confidence that they will fit appropriately.

Triangulation is the process of determining the location of a point by measuring angles to it from known points at either end of a fixed baseline, rather than measuring distances to the point directly (trilateration). The point can then be fixed as the third point of a triangle with one known side and two known angles.

Triangulation can also refer to the accurate surveying of systems of very large triangles, called triangulation networks. This followed from the work of

Willebrord Snell in 1615-17, who showed how a point could be located from the angles subtended from three known points, but measured at the new unknown point rather than the previously fixed points, a problem called resectioning. Surveying error is minimized if a mesh of triangles at the largest appropriate scale is established first. Points inside the triangles can all then be accurately located with reference to it. Such triangulation methods were used for accurate large-scale land surveying until the rise of global navigation satellite systems in the 1980s.

In addition to cartography, optical 3d measuring systems use this principle to determine the spatial dimensions and the geometry of an item. Basically, the configuration consists of two sensors observing the item. One of the sensors is typically a digital camera device, and the other one can also be a camera or a light projector. The projection centers of the sensors and the considered point on the object's surface define a (spatial) triangle. Within this triangle, the distance between the sensors is the base b and must be known. By determining the angles between the projection rays of the sensors and the basis, the intersection point, and thus the 3d coordinate, is calculated from the triangular relations.

With two cameras at known fixed distances, the face of an object in the common field of view of the two cameras can be modeled.

Many people carry a digital camera as a standard feature of their mobile phone. In principle this can be used to visualize an object from two or three positions and the surface topography and size of the object may be calculated. In practice, this requires the relative distance between the two positions to be accurately known, which is rarely the case.

An image obtained by a camera is a 2D rendering of a 3D space that is obtained by a perspective projection onto a virtual viewing surface of the camera that is determined by a viewpoint and viewing ray that are fixed relative to the viewing surface of the camera. The combination of viewing surface, viewpoint and viewing ray determines the perspective of the image.

There are instances when it is useful to photograph a subject or scene from a specific perspective. This may occur, for example, when an accessory to be used with the subject is to be purchased over the Internet, and the supplier requires a photograph of the subject taken from one or more specific perspectives. For example, an individual wishing to purchase clothing items, such as a pair of shoes, over the Internet, may be requested by the vendor to provide one or more photographs of the individual's feet from perspectives specified by the vendor.

### SUMMARY OF THE INVENTION

The present invention is directed to a method, system and software application for using a digital camera, particularly a smart mobile phone, to obtain a size and shape data of a subject such as a foot, for example.

In some embodiments, the data may be transmitted to a supplier for remote purchasing of a complimentary product, such as an article of clothing or a shoe, for example.

In some embodiments, the article of clothing or shoe may be fabricated to fit the subject, or adjusted to fit the subject before dispatching.

A first aspect is directed to a method for obtaining size and shape data of a subject comprising positioning a substantially two dimensional reference object on a plane near to the subject; providing a digital camera; imaging the object and subject on the display screen together with a framework corresponding to a projection of the reference object from a desired angle, and tilting the screen together with a framework corresponding to an outline of the reference object to align the outline with the perimeter of the reference object on the screen.

Typically, the subject and the reference object are viewed from at least two positions, where the edges of the image of the reference object shown on the screen of a digital camera is aligned with a frame shown on the screen to locate the camera in a fixed position and orientation with respect to the reference object.

Typically, the digital camera comprises a display screen, a pixilated array, a processor, a memory and a transmitter.

Optionally, the digital camera is an appropriately programmed smartphone.

Alternatively, the digital camera is a pad computer. In one method, the plurality of positions is two positions. Optionally, the subject is a foot.

Optionally, the reference object is a standard sized sheet of paper. Alternatively, the reference object is selected from the list consisting of a banknote, a business card and a coin.

In one variant method each image is transposed to show the subject from above.

Typically, a plurality of transposed images from the plurality of positions are superimposed.

Optionally, the shape and size of the subject at different elevations is determined.

Optionally, the shape and size of the subject is used for fitting a product to the subject.

Optionally, the product is an article of clothing. It may, however, be a shoe, an insole or a prosthetic. In one of its aspects the present invention provides a method and system for specifying a perspective for viewing on object or scene. In accordance with this aspect of the invention, a 2D reference object is placed on a flat surface within a field of view, such as adjacent to the object or within a scene, for example. The 2D projection of the reference object on a screen of a digital camera depends upon the perspective from which the reference object is viewed. In accordance with this aspect of the invention, a frame is displayed on the camera screen which specifies the projection of the reference object on the camera screen when the reference object is viewed from the specified perspective. A user then positions and orients the camera so that the contour of the projection of the reference object on the camera coincides with the frame. In this manner, the reference object may be viewed from a predetermined, desirable perspective. By viewing the reference object and a nearby subject within the same field of view from two or more of such predetermined perspectives, the surface of the subject may be modeled. In other words, the shape of the subject may be determined.

In another of its aspects, the invention provides a method for generating an image of a surface of a 3D subject object. In accordance with this aspect of the invention, a 2D reference object is placed on a flat surface, and a subject of interest is positioned near to or on the reference object. Two or more images of a scene comprising the reference object and the subject object are obtained from two or more perspectives. Each of the images is rectified using a projective transformation, as explained in detail below. The surface of the subject in contact with the flat surface upon which the subject has been placed can then be obtained by superimposing the rectified images. The aspect of the invention may be used, for example, to construct the image of the planum of a foot from two or more images of the foot taken from different perspectives. The planum of a foot is the surface of the foot facing downwards while standing.

Yet another aspect of the invention is directed to a system for mapping an interior space of a shoe. The system comprises a stereo vision camera that comprises a pair of cameras and a laser pattern projector. The laser pattern projector generates a laser beam that is observed in images obtained by the video cameras and as a spot of light reflected from the inner wall of the interior space of shoe. The stereo vision camera is dimensioned to be inserted into the interior space of a shoe.

The stereo vision camera is connected to the rotor of a motor so that activation of the motor rotates the stereo vision camera. The motor is attached to a horizontal bracket that is supported by a vertical column extending from a base.

The system further comprises a controller that includes a processor and a memory. The processor is configured to activate the motor according to a predetermined time regime and to obtain stereo pairs of images from the stereo camera in each of a plurality of different positions. The obtained stereo pairs of images are stored in the memory.

In use, the stereo camera is inserted into the interior space of a shoe. The controller activates the motor to bring the stereo camera into a predetermined position in the interior space of the shoe, and a stereo pair of images is obtained and stored in the memory. The process is repeated, each time generating a stereo pair of images with the stereo camera in a different predetermined position inside the interior space. In one embodiment, the stereo camera is rotated by a small angle θ between obtaining consecutive stereo pairs of images until the camera has performed a complete rotation.

After collection of the stereo pairs of images, the location (pixel address) of the laser spot in each image in a stereo pair of images is determined. From the pair of locations, the path length of the laser beam from the orientation of the stereo camera to the inner wall of the interior space is obtained from the calibration data. A three dimensional model of the interior space can then be constructed.

In another of its aspects, the invention provides a system for mapping an interior space of a shoe. The system comprises a stereo vision camera that is dimensioned to be inserted into the interior space of a shoe and a laser pattern projector affixed onto the camera. The laser pattern projector generates a laser beam that is observed in images obtained by the stereo camera as a spot of light reflected from the inner wall of the interior space of shoe.

The stereo camera is inserted into the interior space of a shoe, and a plurality of images of the interior space is obtained, each time with the camera facing a different direction. A motor is used to bring the stereo camera into a predetermined position in the interior space of the shoe, and a stereo pair of images is obtained and stored in a memory. After collection of the stereo pairs of images, the location (pixel address) of the laser spot in each image in a stereo pair of images is determined. From the pair of locations, the path length of the laser beam from the orientation of the stereo camera to the inner wall of the interior space is obtained from calibration data. A three dimensional model of the interior space can then be constructed.

### BRIEF DESCRIPTION OF FIGURES

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice. In the accompanying drawings:

FIG. 1 is a flowchart of a generalized method of the invention;

FIG. 2 is a functional block diagram of the digital camera;

FIG. 3 is a photograph of a foot standing on a piece of A4 paper, such that the angle of the photograph distorts the image of the rectangular paper into a trapezoid.

FIG. 4 is a second photograph of the foot and piece of A4 paper shown in FIG. 3, taken from a second viewing angle.

FIG. 5a shows a bare foot standing on an A4 piece of paper as imaged on a screen of a digital camera, and a projection of a reference frame on the screen of the digital camera.

FIG. 5b shows the bare foot standing on an A4 piece of paper of FIG. 5a, but with the screen of the digital camera manipulated to bring the projection of the A4 sheet into alignment with the reference frame on the screen of the digital camera.

FIG. 6 shows how the projections of two (or more) images of a foot may be superimposed to extract the planum of the foot.

FIG. 7a shows the planum of a foot aligned with the internal dimensions of a shoe that is too tight.

FIG. 7b shows the planum of a foot aligned with the internal dimensions of a shoe that is a perfect fit.

FIG. 7c shows the planum of a foot aligned with the internal dimensions of a shoe that is too loose.

FIG. 8 is a schematic illustration of foot, showing how a planum can provide an indication of the shape of the foot in three dimensions.

FIG. 9 is a virtual 3d last corresponding to the foot.

FIG. 10a shows how a subject of interest, here a foot, may be positioned along-side a coin which may serve as a reference object to calculate the viewing angle and distance of a viewpoint by the distortion of the projection of the coin from a circle.

FIG. 10b shows how a subject of interest, here a foot, may be positioned along-side a bank note which may serve as a reference object to calculate the viewing angle and distance of a viewpoint by the distortion of the projection of the bank note from a rectangle.

FIG. 11 is a flow chart of a method for obtaining images of a reference object and a subject of interest from specific viewing perspectives and for taking the specific viewing perspectives obtained by the method of FIG. 11a and using them to extract a planum of the subject, here the foot.

FIG. 12 is a schematic illustration of a foot standing on a sheet of A4 paper and two viewing perspectives X and Y.

FIG. 13 is a schematic illustration of the foot of FIG. 12 from a perspective X.

FIG. 14 is a schematic illustration of the foot of FIG. 12 from perspective Y.

FIG. 15 is a flow chart of a method for selecting a list corresponding to a planum, and

FIG. 16 is a schematic illustration of a system for tracking an inside surface of a cavity such as a shoe.

### DESCRIPTION OF PREFERRED EMBODIMENTS

Embodiments of the present invention use tools such as image processing, computer vision, optimization algorithms on mobile platforms and complex algorithms processing controlled by mobile platforms that are sometime supported by cloud infrastructure to obtain size and shape data for subjects of interest, particularly body parts for ordering shoes and clothing from a supplier. This enables the correct sizes of such shoes and clothing to be ordered from a catalog or a website, and in some instances, may be used to have such shoes and clothing made to fit.

A particular feature of preferred embodiments of the present invention uses the distortion of size and shape of a reference object shown on the screen of a digital camera to position that camera at a known viewing angle and distance from the reference object.

The distance and angle is used to calculate the angles of points on the surface of the subject within the field of view. Using a plurality of images, which may be two or more, the topography of the surface of the subject is calculated.

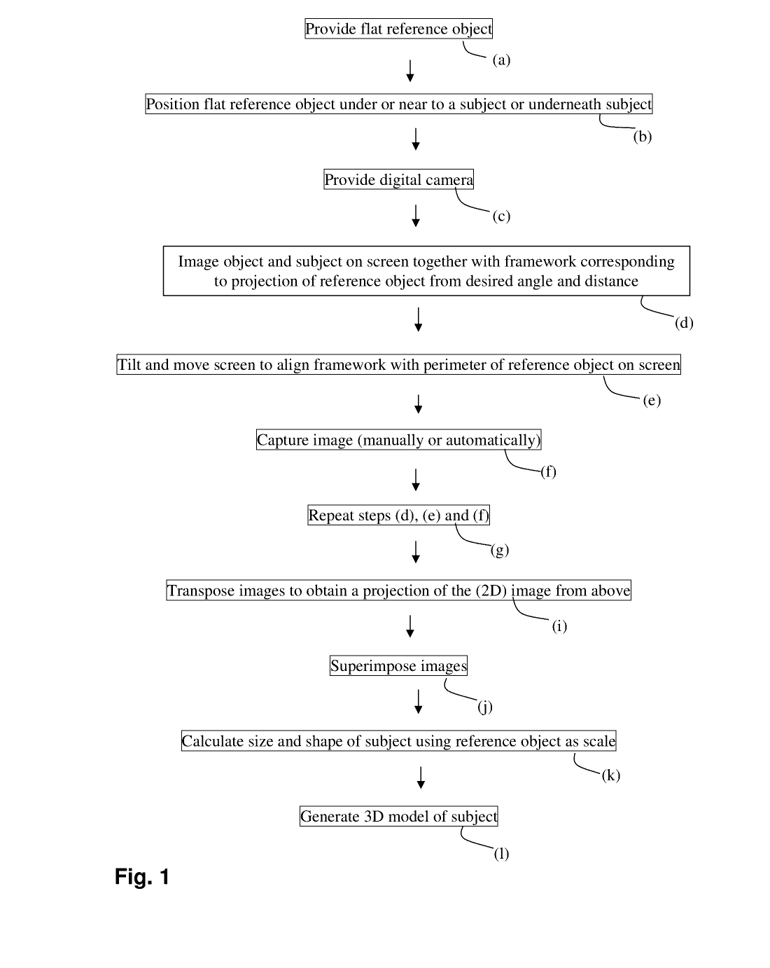

With reference to FIG. 1, a method for obtaining size and shape data of a subject is described. Firstly, a substantially two dimensional reference object is provided—step (a) and is positioned near the subject to be measured—step (b), preferably on the horizontal plane on which the subject is positioned, thereby ensuring that its position with respect to the subject is known. A digital camera is provided—step (c). The digital camera is directed at the object and subject such that they are displayed on the display screen of the digital camera together with a framework corresponding to a projection of the reference object from a desired angle—step (d). The screen, together with a framework corresponding to an outline of the reference object is moved and tilted to align the outline with the perimeter of the reference object on the screen—step (e). The image is captured—step (f). The procedure is repeated from at least one additional position, so that the subject and the reference object are viewed from at least a second position where the edges of the image of the reference object shown on the screen of a digital camera is aligned with a second frame shown on the screen to locate the camera in a further position and orientation with respect to the reference object and a further image is taken—step (g).

For simplicity, the reference object is preferably a standard size and shape and is substantially flat. A known coin or bank note may be used. Credit cards are an alternative since these have standard size or shape. It will be appreciated that in general, the larger the reference object the more pixels it covers on the screen of the digital camera. This helps provide accuracy in determining the position of its corners. In a preferred embodiment, a standard sized sheet of paper is used. For most of the world, ISO standard sizes are used and A4 is very commonly used for office printers and is fairly ubiquitous. In North America, there are other standard sizes such as Legal (American Foolscap), letter, etc.

In all standard sizes of paper sheets, the sheet is rectangular. If a piece of paper is placed flat on a horizontal surface such as a floor and viewed through a digital camera from various angles of elevation of viewing positions, the image of the rectangular sheet as viewed on the screen of the digital camera is transformed into a quadrilateral that may be a trapezoid with the nearest side being parallel and shorter than the furthest side, and the connecting sides converging towards a vanishing point. The shape and size of the paper as viewed on the camera screen may be used to calculate the distance, viewing angle including the angle of elevation.

In one embodiment, a reference frame corresponding to the distorted quadrilateral image of the sheet of paper from a predetermined position (angle, distance and angle of elevation) is displayed on the screen. The user manipulates the camera by tilting the screen to bring the image of the sheet of paper (or other predetermined reference object) into alignment with the frame—step (e) and the subject is photographed, i.e. the image is captured—step (f).

This is repeated for at least one further position by displaying a second frame having a shape and size on the screen and manipulating the camera to bring the edges of the image of the reference object into alignment with the frame, and the further image is captured—step (g).

Each image may be easily transformed to view the subject from above such that a rectangular reference object such as a piece of paper is seen as a rectangle—step (i). Indeed, using appropriate transformations, the appearance of the reference object and the subject may be transposed to any other position vis a vis the actual position. If, say, a coin is used as the reference object, the ellipse viewed may be transformed back into a circle as if viewed from above. A series of reference coordinates may be used. These can be Cartesian or polar. Matrices may be used for the transformations.

If a subject is viewed from two or more positions, the images may be transposed to superimpose the images—step (j).

The size and shape of the subject may be calculated using the size of the reference object as a scale—step (k).

Many subjects have a general shape and size. So, for example, knowing that a subject is an egg or a foot, helps generate a reasonable model of its surface with relatively few data points.

Knowledge of the shape and size of the subject may be used to simplify the calculations for generating a reasonable model.

A three dimensional model of the subject may be generated—step (l).

Such a model may be used to select and fit a product to the subject.

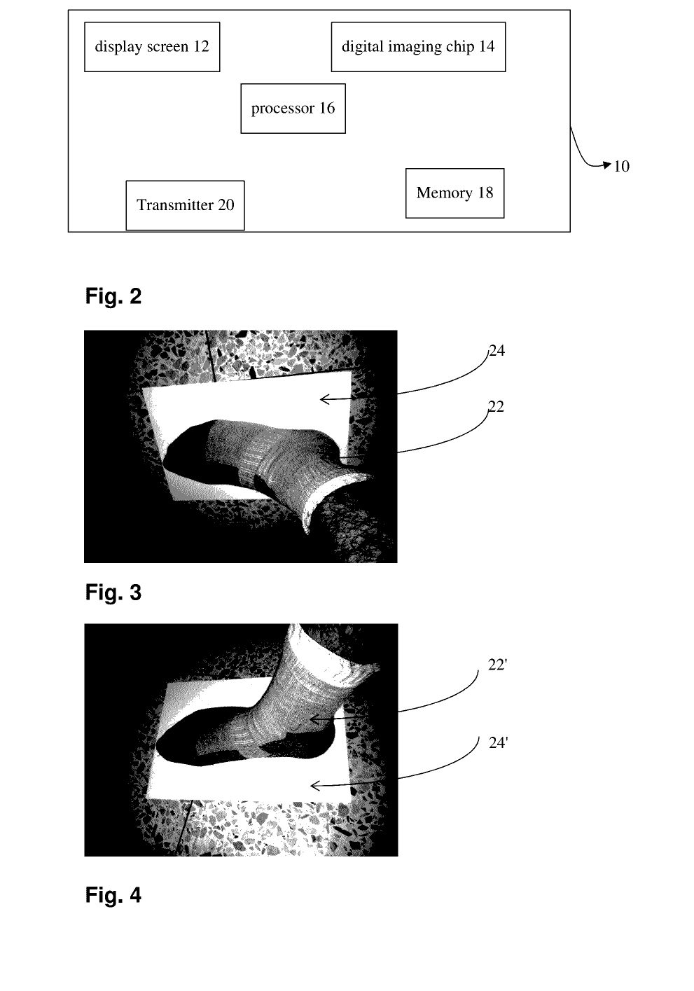

With reference to FIG. 2, the digital camera 10 comprises a display screen 12, a digital imaging chip 14, a processor 16, a memory 18 and a transmitter 20. It will be appreciated that a mobile phone, particularly a smart-phone typically includes the desired components. Smart phones may thus be programmed to create a three dimensional model of a foot or other subject by creating an appropriate application to execute the method of claim 1.

With reference to FIG. 3, by way of example, a foot 22 may be selected as the subject and positioned on an A4 piece of paper, used as a reference object. FIG. 3 is the image of the foot shown on the screen of a digital camera 10, such as a smart phone, for example.

It will be noted that although the sheet of A4 is rectangular, due to perspective effects, the nearer edges appear longer than the further edges and the rectangle is seen as an irregular quadrilateral. Where the camera is held such that one edge is parallel with an edge of the paper, the quadrilateral appears to be trapezoidal.

With reference to FIG. 4, the same foot 22′ may be imaged from a different position. The paper will be distorted into a different quadrilateral 24′.

In general, the subject of interest (such as a foot) together with the reference object (such as a sheet of paper) may be imaged from a plurality of positions and each image may be transposed into an image of the subject from above at the same scale.

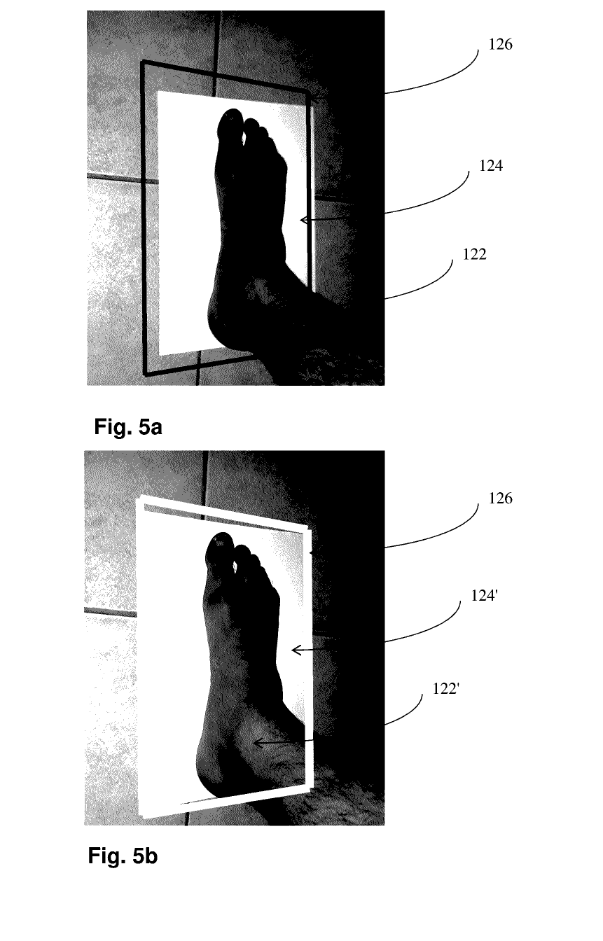

Referring to FIG. 5a, a photograph or screen capture corresponding to the image displayed on the screen 12 of a digital camera 10 is shown. The photograph includes an image of a subject foot 122 standing on an image of a reference piece of paper 124 and also shows a quadrilateral frame 126. The quadrilateral frame 126 has a different shape and size than the image of the reference piece of paper 124 and cannot be aligned with it to an acceptable degree. Referring to FIG. 5b, by moving the camera closer and further from the foot 122′, the image of the reference object paper 124′ may be better sized to the frame 126 on the screen. By adjusting the angle of tilt, the shape of the paper 124′ may as seen on the screen may be adjusted to correspond with the frame 126. Thus using the frame 126 as a guide, the position of the digital camera 10 may be adjusted to view the sheet of paper 124 and the foot 122 from a predetermined position.

It will be appreciated that the process of determining the right capturing position may be fully automatic. Once the frame of the reference object is displayed on the user's screen, and once the displayed frame is close enough to the real contour of the real reference object—images may be taken automatically by the digital camera, without the user having to manually depress the photograph button that is analogous to operating the shutter of a conventional camera. The process of edge detection and a comparison between the edges image to the reference frame can be used to automatically capture the image.

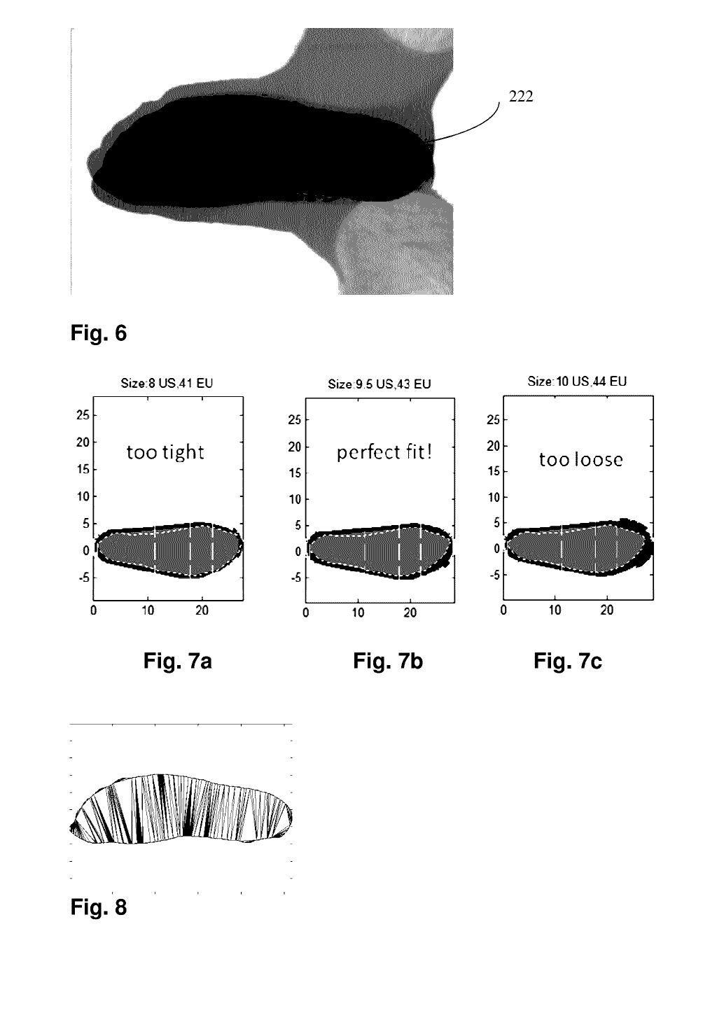

With reference to FIG. 6, a foot imaged from two positions, in this case, the images 22 and 22′ shown in FIGS. 4 and 5 respectively, may be superimposed to give the image 222. This is, itself, a useful indication of the planum of the foot and may be used to calculate the length and width of the foot, to select an appropriate shoe size. It will be appreciated, however, that different styles of shoes having the same shoe size and width may have very different shapes and may be more or less appropriate for being worn on different shaped feet. As shown, the surrounding rectangle is A4 size so the image may be printed onto paper using an office printer and used as a simple template to check against the sole of a shoe or printed onto card and inserted into a shoe to check the fit. With reference to FIGS. 7a, 7b and 7c, the image 222 (rotated through 180°) can be positioned onto the sole of a shoe in a variety of sizes and the correct size, in this case 9.5 US (43 EU) is selected. Since different countries use different scales and these do not line up exactly, this is very useful.

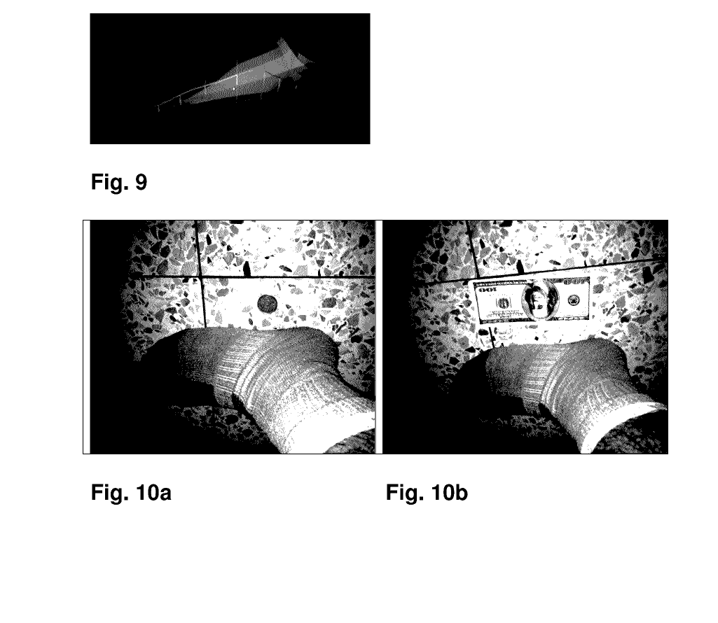

Furthermore, with reference to FIGS. 8 and 9, a virtual three-dimensional computer model of the subject foot may be generated. A reasonable three dimensional model may be generated from two viewing positions. Additional viewing positions can provide details of back surfaces. For example viewing a foot from the left, right and somewhere to the rear enables the foot to be well modeled, including the ankle, toe, in-step and outer surface, whereas two points of view would be less satisfactory. Additionally, if three or more points are used, an average position (or weighted average) may be used to more accurately model the object of interest.

Thus a shoe size may be selected for a particular foot. This enables online or catalog purchasing of shoes. Using a similar technique to model other limbs and body parts, other items of clothing, such as gloves, hats, and the like, may be ordered online with an increased likelihood of a correct fit and a corresponding reduced likelihood of return. In addition to economies of scale, since a supplier may sell directly to more customers, the possibility of supplying different sized shoes that better fit the two feet becomes economically feasible. It also makes making shoes or adjusting them in the factory to fit a customer possible.

Using an augmented reality based visualization technique the raw captured images of the subject body part such as the foot, for example, may be over layered with a projected model of the desired article of clothing, such as a shoe, etc.

Smart phones often include tilt sensors. These can be used to help orientate the smart phone to bring the edges of the image of object on the screen into alignment with a frame displayed thereon by displaying a number indicating how close one is to the correct tilt.

To help align the perspective view of the shape of the reference object on the camera screen with the image of the reference object shown on the screen, a tilt angle may be shown on the screen. The tilt angle shown may be the actual tilt angle of the screen as calculated using sensors such as gyroscopic sensors and accelerometers in the digital camera, and/or the desired tilt angle. Typically, both are shown or a required adjustment to the actual tilt to bring the two images into alignment is shown. This is particularly appropriate when using the digital camera and the screen of a mobile phone for imaging the subject and calculating its size. It will be appreciated that the digital camera needs to be correctly angled in two directions. Two number readings may be used to facilitate this.

With reference to FIGS. 10a and 10b, other standard objects such as a coin or bank note may be used a reference object.

Not only may a smart-phone conveniently be used to obtain two or more images of a subject and to calculate the dimensions of the subject and to create a virtual three dimensional model of the subject, but, using its transmission capabilities, typically its messaging, mail or internet, functionality, this data may be transmitted to a supplier of a complimentary object. Thus for example, a smart phone may be used to photograph a foot from a plurality of preset relative positions. Imaging a foot standing on a sheet of paper from above can help selecting the right size shoe. The data obtained may be transmitted to a website for purchasing shoes, for example.

It will be appreciated that by photographing from above, only the upper surface of the foot is seen and the sole of the foot is not seen, but the topography of the upper surface of the foot may provide a good indication of whether a subject foot is flat footed, and whether a particular type of shoe, such as a stiletto heal is appropriate.

In a variant method, a sheet of translucent material, preferably substantial transparent material is pressed against the foot and the foot is imaged through the sheet. If a reference is shown on the sheet, the shape and size of the subject may be extracted.

For example, if a rectangular framework of a standard size such as A4, for example, is marked onto a sheet of transparent material, and the sheet is pressed against a foot, a single image from a single point may provide some information regarding whether the foot is flat footed, and whether insoles are required.

If this single image is combined with additional information regarding the shape of the foot such as the virtual model from above and knowledge of physiology, particularly podiatry, the shape of the sole and the arch may be calculated more accurately.

Alternatively, a subject foot may be brought into contact with a touch screen of an iPad to generate information regarding the footprint, from which further information may be derived.

If the sole of a foot is imaged from two or three spots, its topography, which is the shape of an appropriate insole, may be modeled, or mapped onto a coordinate system, using similar algorithms to those used for modeling the outside of the foot.

This may be effected by placing a sheet of a transparent polymer with a scale marked thereonto, such as an A4 sized frame drawn thereonto, against the sole of the foot, and using the distortion of the A4 frame into a non-square quadrilateral to position a digital camera at a known angle and distance to calculate the shape and size of the sole of the foot.

Since human feet have a shape that follows a well known pattern, it is possible to obtain less information from photographic techniques and to use extrapolation to create a model that can be used to select appropriate footwear.

By way of example, specific embodiments are now discussed, with respect to imaging a foot.

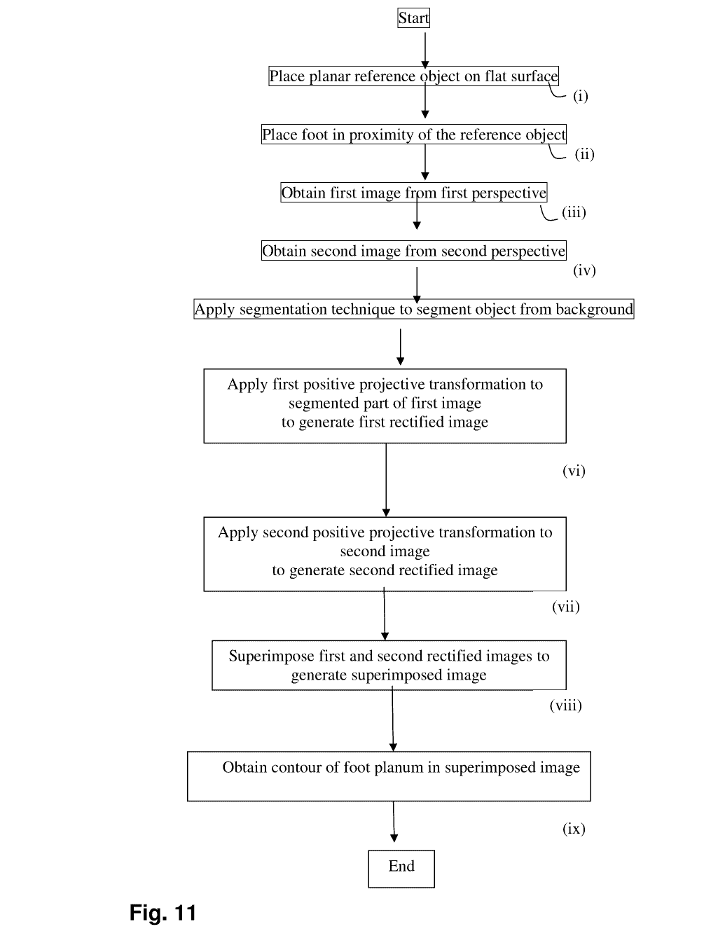

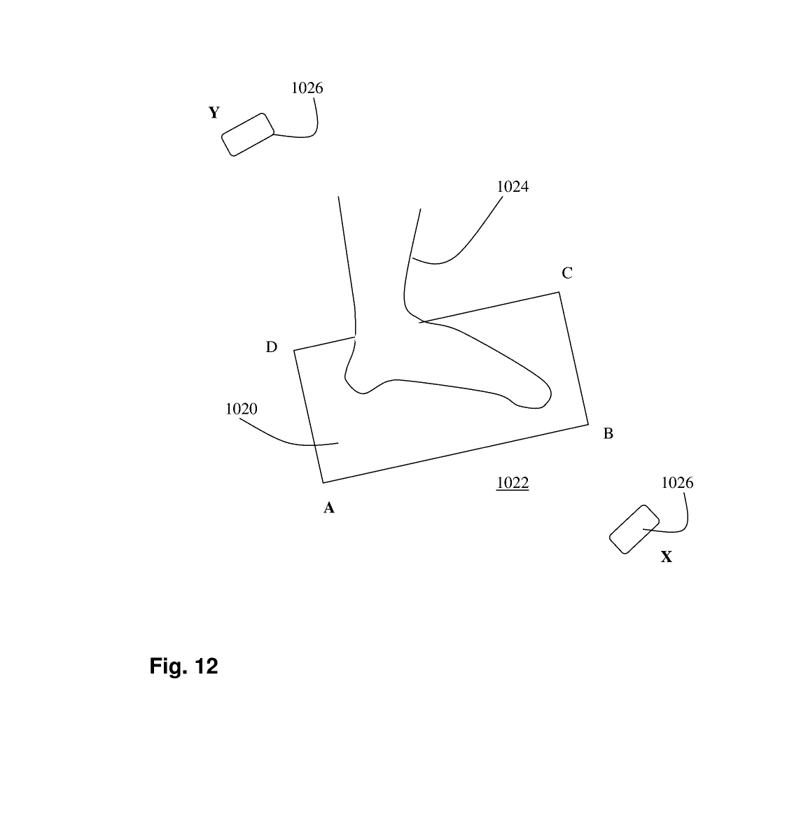

FIG. 11 shows a flowchart illustrating a method of obtaining an image of a foot from a specified perspective, in accordance with one embodiment of this aspect of the invention. Firstly, a planar reference object 1020 (see FIG. 12) of known dimensions is placed on a flat surface, such as a floor 1022—step (i). The reference object 1020 may be, for example, a sheet of paper of known shape and dimensions, or a banknote of known shape and dimensions. The foot 1024 to be imaged is then placed on or near the reference object—step (ii). Only the foot 1024 is shown in the scene depicted in FIG. 12, the remaining body parts having been omitted for the sake of clarity. The foot 1024 to be imaged may be bare or may be within a sock or stocking.

A first image of the scene, including the foot 1024 and the reference object 1020, is then obtained from a first perspective using a camera 1026—step (iii). The reference point X shows the camera 1026 when positioned in space so as to obtain a first image from the first perspective. The first perspective is selected so that the reference object 1020 is not viewed from directly above in the first image. This occurs when the viewing surface of the camera 1026 is angled with, or not parallel to the surface 1022. FIG. 13 shows the perspective projection of the scene 1030 as might appear on a screen 1028 of the camera 1026, when the scene is viewed from the first perspective (position X). Since the reference object 1020 is not viewed from directly above in the first image, the reference object 1020 will appear distorted in the first image. For example, if the reference object ABCD 1020 is rectangular in shape, then in the perspective projection of the scene shown in FIG. 13, the reference object 1020 may appear to be trapezoidal A′B′C′D′ in shape.

The first perspective of FIG. 13 may be specified to a user by displaying on the screen 1028 of the camera 1026 a frame indicating the shape and size of the perimeter of the reference object 1020 when the scene 1030 is viewed from the first perspective. The user manipulates the camera 1026 and positions the camera 1026 in the scene so that the perimeter of the reference object 1020 on the screen 1028 is bordered by the frame, and once the camera 1026 is thereby correctly positioned in a first predetermined position, and tilted to a predetermined viewing angle, the user takes a first digital photograph which is essentially a screen capture of that viewed on the display screen 1028 of the camera 1026 (which may be a smart-phone), thereby obtaining the first image. See FIGS. 5a and 5b to see how the frame 126 may be aligned with a reference sheet of paper 124 by tilting in two directions.

Although in other embodiments, other reference objects may be used, a sheet of paper 124 is particularly suitable as it has four clearly and unambiguously defined corners which serve as fixed co-planar reference points, of which no three are mutually colinear.

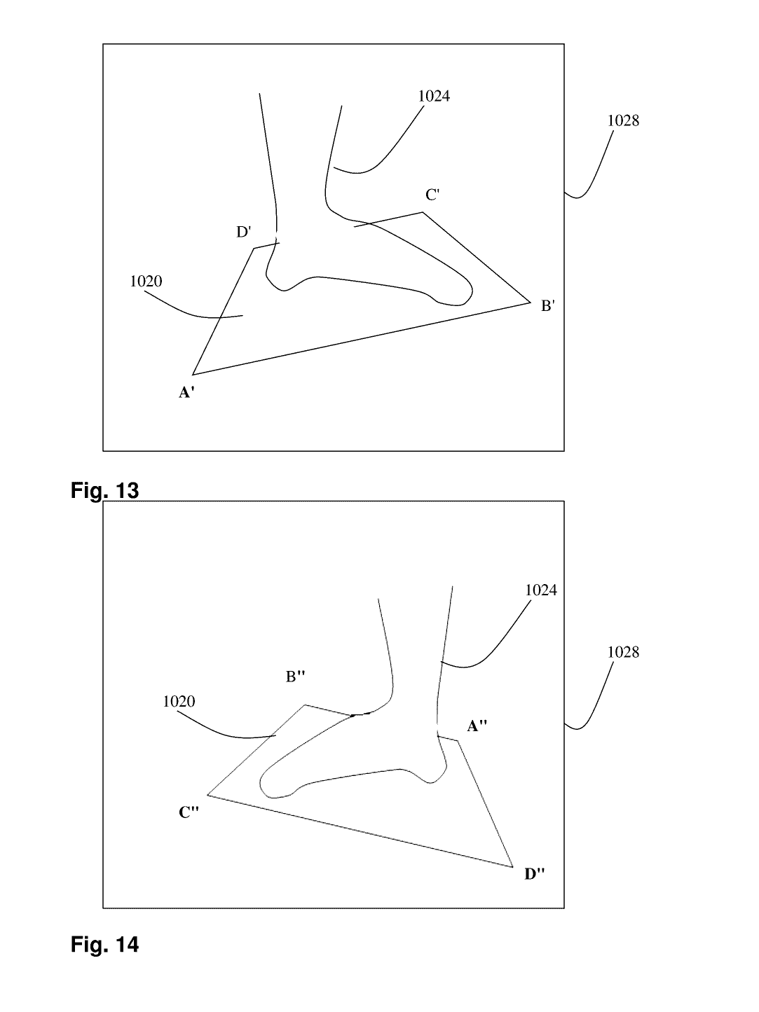

A second image of the scene may be desired from a second perspective (step (iv). The reference Y in FIG. 12 shows the camera 1026 when positioned in space to obtain the second image from the second perspective. As with the first perspective, the second perspective is selected so that the reference object 1020 is not viewed from directly above in the second image, and this occurs when the viewing surface of the camera 1026 is not parallel to the surface 1022. FIG. 14 shows the perspective projection of the scene as it might appear on the screen 1028 of the camera 1026, when the scene is viewed from the second perspective (Y). Since the reference object 1020 is also not viewed from directly above (en face) in the second image, the reference object 1020 appears distorted in the second image.

The second perspective may be specified to a user by displaying on the screen 1028 of the camera 1026 a frame 1034 indicating the contour of the reference object 1020 when the scene is viewed from the second perspective. The user manipulates the camera 1026 and positions the camera 1026 in the scene so that the image of the reference object 1020 on the screen 1028 is bordered by the frame 126 (FIG. 5), and obtains the second image.

Additional images of the scene from additional perspectives may be obtained in a similar fashion; the perspective of each image being specified to the user by displaying a frame on the camera screen indicated the 2D projection of the reference object on the screen from the specified perspective.

Two or more images of the foot 1024, obtained from two predetermined positions by the camera 1026, using the frame displayed on the camera screen to specify the viewing angle and distance of the camera 1026 from the same reference object 1020, may be used to generate an image of the planum of the foot. The planum of a foot is the surface of the foot facing downwards while standing. FIG. 11b shows a flow chart for a method of imaging a planum of a foot. A first image of the foot obtained by the method of FIG. 11, is segmented from its background (step v) and the segmented image is then subjected to a first projective transformation—step (vi) to generate a first rectified image. The first projective transformation is the unique projective transformation that maps the contour of the reference object 1020 in the first image onto the contour of the reference object 1020 when the reference object 1020 is viewed from directly above (en face). Thus, for example, if the reference object 1020 has a rectangular shape having vertices A, B, C, and D (FIG. 12), the first projective transformation will map the contour of the reference object 1020 in the first image onto the contour of the reference object when viewed en face by mapping the vertices A′, B′, C′, and D′ (FIG. 3) onto the vertices A, B, C, and D, respectively.

Now a second image is subjected to a second projective transformation (step (vii) to generate a second rectified image. The second projective transformation is the unique projective transformation that maps the contour of the reference object 1020 in the second image onto the contour of the reference object 1020 when the reference object 1020 is viewed en face. Thus, if the reference object 1020 is has a rectangular shape having vertices A, B, C, and D (FIG. 2), the second projective transformation will map the contour of the reference object 1020 in the second image onto the contour of the reference object when viewed en face by mapping the vertices A″, B″, C″, and D″ (FIG. 14) onto the vertices A, B, C, and D, respectively.

Referring to step (viii), the first and second rectified images are superimposed upon one another to generate a superimposed image. The first and second rectified images are superimposed upon one another in such a way that the vertices A, B, C, and D in the first rectified image are mapped into the vertices A, B, C, and D in the second rectified image, respectively.

The contour of the planum of the foot 24 is then extracted from the superimposed image—step (viii). The contour of the planum may be, for example, the contour of the region in the superimposed image where the images of the foot 1024 in the first and second rectified images overlap. The process then terminates. A boundary extraction program may be used to extract the shape of the planum.

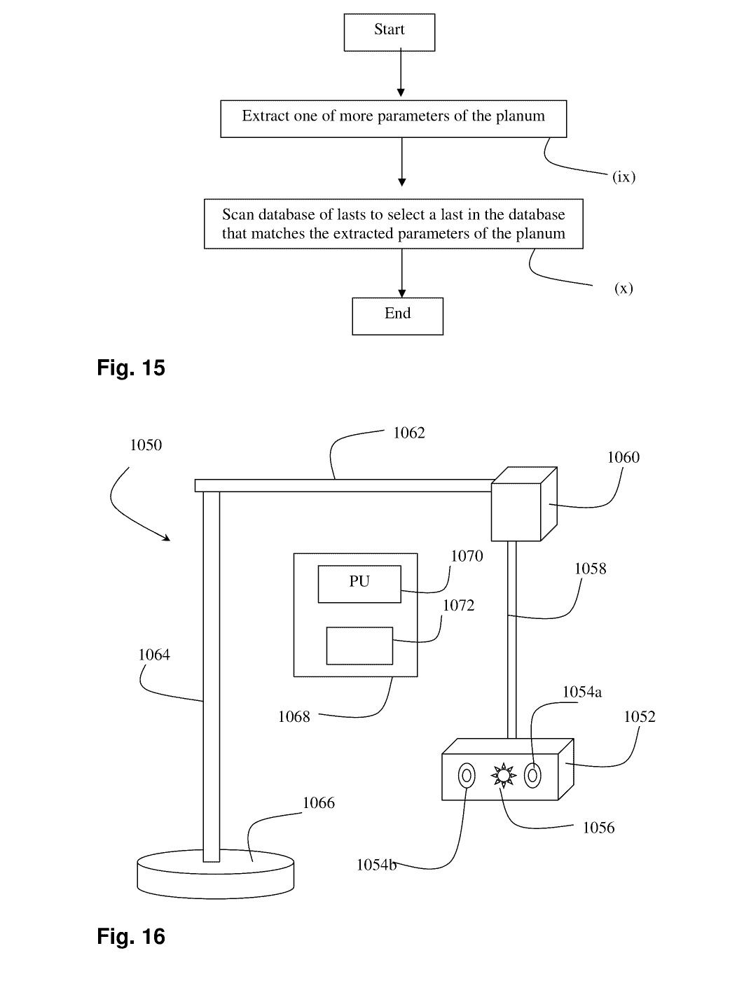

As mentioned hereinabove, simply knowing the shape of the planum, i.e. the shape of the foot that would be obtained by placing the foot on a flat surface and drawing around the foot is sufficient to enable a reasonable choice of shoe with increased likelihood of it fitting well and resultant customer satisfaction. However, the method described hereinabove may be used to obtain much more data about the shape of the foot, particularly since human feet, though differing from person to person, tend to fall into well established categories of feet type and so a virtual model of the foot, or, if one prefers, a virtual last may be created. In another of its aspects, the invention provides a system and method for selecting a virtual last corresponding to a planum, such as the planum obtained by the method of FIG. 11. FIG. 15 shows a process for selecting a virtual last corresponding to a planum, such as the planum obtained by the method of FIG. 11. In step (ix), one or more parameters of the planum are extracted. The extracted parameters may include, for example, any one or more of arch height, planar arch width, rearfoot angle, arch angle, arch index, Chippaux-Smirak index, Staheli Index, and the toe type (e.g. Egyptian type, Greek type, or square type). Definitions of the various planum parameters may be found, for example, in Science of Footwear, by R. S. Goonetilleke, Boca Raton, Fla., CRC Press, 2013, 726 pp., pages 23-29.

Next in step (x), a database of virtual lasts is searched for a last having parameters that best match the parameters that were extracted from the planum, and the process ends. Once one or more virtual lasts have been found for the planum, shoes may be found having an interior space corresponding in shape to the shape of the virtual last. This is performed by extracting one or more parameters of a last, and scanning a database of shoe interior spaces for shoes having an interior space having parameters corresponding to the parameters of the last. The extracted parameters of the last may be for example, any one or more of the bimalleolar width, the ball girth, minimum arch girth, heel girth, the medial or lateral malleolus height, the dorsal arch height, the ball angle, the hallux angle, and the digitus minimus angle. Definitions of the various last parameters may be found, for example, in Science of Footwear, by R. S. Goonetilleke, Boca Raton, Fla., CRC Press, 2013, 726 pp., pages 23-29.

It will be appreciated that a physical last corresponding to a virtual last could easily be fabricated, by digital printing for example. This could be used to order shoes on line and to supply foot dimensions, so that a factory can fabricate made to measure shoes. This is generally not required though. Even without manufacturing to order, having information regarding the true size and shape of each foot can be used to select appropriate shoes. Also, different sized shoes can be purchased for each foot. Conventionally, with physical shoe shops, this was not feasible, but online purchasing enables selecting shoes from higher up the retail chain and possibly from the factory.

A further aspect of the invention provides a system for mapping an interior space of a shoe. This enables categorizing shoe types as appropriate for feet types. This may be achieved by mapping the interior space of a shoe. With reference to FIG. 16, a system 1050 for mapping an interior space of a shoe is shown. The system 1050 comprises a stereo vision camera 1052. The stereo vision camera comprises a pair of cameras 1054a, 1054b, and a laser pattern projector 1056. The laser pattern projector 1056 generates a laser beam that is observed in images obtained by the video cameras 1054a, 1054b as a spot of light reflected from the inner wall of the interior space of shoe. The stereo vision camera 1052 is dimensioned to be inserted into the interior space of a shoe.

The stereo vision camera 1052 is connected to a spindle (rotor) 1058 of a motor 1060 so that activation of the motor 1060 rotates the stereo vision camera 1052. The motor 1060 is attached to a horizontal bracket 1062 that is supported by a vertical column 1064 extending from a base 1066.

The system 1050 further comprises a controller 1068 that includes a processor 1070 and a memory 1072. The processor 1070 is configured to activate the motor 1060 according to a predetermined time regime and to obtain stereo pairs of images from the stereo camera 1052 with the stereo camera 1052 in each of a plurality of different positions. The obtained stereo pairs of images are stored in the memory 1072.

The stereo camera 1052 may be calibrated by inserting it into an enclosed space of known shape and dimensions and obtaining a plurality of stereo pairs of images, as explained below. The position (pixel address) of the laser spot from the laser pattern projector 1056 in each image in a stereo pair of images is correlated with the known path length of the laser beam from the stereo camera 1052 to the inner wall of the interior space. A separation of the cameras 1054a and 1054b of about 50 mm allows an accuracy of +1 mm in a measurement of 150 mm in front of the stereo camera 1052.

In use, for mapping the inside surface of footwear, (such as a shoe or boot), the stereo camera 1052 is inserted into the interior space of the footwear. The controller 1068 activates the motor 1060 to bring the stereo camera 1052 into a predetermined position in the interior space of the footwear, and a stereo pair of images is obtained and stored in the memory 1072. The process is repeated a plurality of times, each time generating a stereo pair of images with the stereo camera 1052 in a different predetermined position inside the interior space. In one embodiment, the stereo camera 1052 is rotated by a small angle θ between obtaining consecutive stereo pairs of images until the camera has performed a complete 360° rotation.

After collection of the stereo pairs of images, the location (pixel address) of the laser spot in each image in a stereo pair of images is determined. From the pair of locations, the path length of the laser beam from the orientation of the stereo camera 52 to the inner wall of the interior space is obtained from the calibration data. A three dimensional model of the interior space can then be constructed.

Although described herein with respect to measuring and modeling feet for selecting the correct model and size of foot-ware, it will be appreciated that the same concepts may be used to determine the shape of other body parts, such as the shape and size of the hand to fit a glove, the shape and size of the breast to fit an appropriate brassiere, the shape of the buttocks and thighs to fit a skirt or slacks or the shape of the face to fit a mask.

It will be noted that whilst breasts do not have a flat surface analogous to the planum of a foot, their shapes and size do, nevertheless, follow well known patterns and there a brassiere can be selected by knowing the general girth or mass of each breast which determines the cup size, and by knowing the overall dimension around the bust which determines the bra size.

The same methodology could be used to design and fabricate prosthetics, such as a false leg or arm to a stump.

Traditional retailing is losing popularity and market share to internet purchasing from catalogues and websites. By a customer imaging a foot in the herein described manner, a shoe seller may check that an ordered size and style of footwear is appropriate or can help the customer select the appropriate size.

Since clothing and footwear sizes do vary from country to country but Internet purchases may be from anywhere in the world, the above method may help ensure that a purchased article of clothing or footwear does, indeed fit.

Furthermore, a hem or trouser leg may be shortened or lengthened before dispatching or an article of clothing may be otherwise altered or finished. Indeed, a three dimensional model of the foot (virtual last) could be used to fabricate a made-measure shoe by transmitting these dimensions to a factory. Optionally the virtual model of a foot could be used to fabricate a last that could then be used to manufacture a close-fitting shoe, for example.

Apart from opening up made-to-measure possibilities, it is well known that most people have one foot that is larger than the other. With relatively small amounts of shoes of a particular design and colour being sent to a retail outlet, traditionally shoes are sold in pairs of the same size. Invariably one shoe is too tight or one shoe is too loose. Since the present invention enables mail order from higher up the supply chain, it becomes economically feasible to order right and left shoes in different sizes, ensuring a more comfortable and supporting fit.

Although described hereinabove with reference to shoes, it will be appreciated that invention may be used to purchase other items of clothing such as gloves, shirts, trousers and hats, for example. Not only could a brassiere be made to measure, but breast enhancers, prosthetic breast inserts for use after a mastectomy could be fabricated.

Thus persons skilled in the art will appreciate that the present invention is not limited to what has been particularly shown and described hereinabove. Rather the scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1. A method for matching an article of footwear to a foot comprising:
(a) obtaining size and shape data of a foot by positioning a substantially two dimensional reference object on a plane near to the foot;
providing a digital camera;
imaging the object and foot on the display screen together with a framework corresponding to a projection of the reference object from a desired angle, and tilting the screen together with a framework corresponding to an outline of the reference object to align the outline with the perimeter of the reference object on the screen,
(b) comparing the size and shape data of the foot with a mapping of the inside surface of a data-base of articles of footwear, wherein each inside surface mapping is obtained the steps of inserting the stereo vision camera of claim into the interior space of the footwear, activating a motor to bring the stereo camera into a predetermined position in the interior space of the footwear, taking stereo pairs of images and stored them within the memory, repeating the process to generating further stereo pairs of images for additional positions of the stereo vision camera inside the interior space, and creating a three dimensional model of the inside of the article of footwear. A method for obtaining size and shape data of a subject comprising
positioning a substantially two dimensional reference object on a plane near to the subject;
providing a digital camera;
imaging the object and subject on the display screen together with a framework corresponding to a projection of the reference object from a desired angle, and tilting the screen together with a framework corresponding to an outline of the reference object to align the outline with the perimeter of the reference object on the screen.

2. The method of claim 1, wherein the subject and the reference object are viewed from at least two positions, where the edges of the image of the reference object shown on the screen of a digital camera is aligned with a frame shown on the screen to locate the camera in a fixed position and orientation with respect to the reference object.

3. The method of claim 1, wherein the digital camera comprises a display screen, a pixilated array, a processor, a memory and a transmitter.

4. The method of claim 1, wherein the digital camera is an appropriately programmed smart-phone.

5. The method of claim 1, wherein said plurality of positions is two positions.

6. The method of claim 1, wherein images of the subject from the plurality of positions is used to calculate size and shape of the subject.

7. The method of claim 1, wherein the digital camera is a pad computer.

8. The method of claim 1, wherein the reference object is selected from the list of a standard sized sheets of paper, banknotes, playing cards, business cards and coins.

9. The method of claim 1, wherein each image is transposed to show the subject from above to extract a planum.

10. The method of claim 1, wherein a plurality of transposed images from the plurality of positions are superimposed.

11. The method of claim 1, wherein the shape and size of the subject at different elevations is determined.

12. The method of claim 1, wherein the shape and size of the subject is used for fitting an accessory to the subject.

13. The method of claim 1, wherein the accessory is footware and the subject is a foot.

14. The method of claim 1, wherein the article of clothing.

15. The method of claim 1, wherein the accessory is a prosthetic.

16. The method of claim 1, wherein the accessory is an insole.

17. A system for mapping an interior space of a container comprising:
a stereo vision camera that comprises a pair of cameras, and
a laser pattern projector for generating a laser beam that is observable in images obtained by the video cameras as a spot of light reflected from an inner surface of the container.

18. The system of claim 17 wherein the stereo vision camera is dimensioned to be inserted into a foot cavity of an article of footwear.

19. The stereo vision camera of claim 17 coupled to a spindle (rotor) of a motor for rotating the stereo vision camera.

20. The system of claim 19 wherein the motor is attached to a horizontal bracket that is supported by a vertical column extending from a base.

21. The system of claim 20 further comprising a processor and memory, the processor configured to activate the motor according to a predetermined time regime and to obtain stereo pairs of images from the stereo camera with the stereo camera in each of a plurality of different positions, and the memory configured to store the obtained stereo pairs of images.

22. The system of claim 20, wherein the stereo camera may be calibrated by insertion into as cavity of known shape and size, and by obtaining a plurality of stereo pairs of images.

23. A method for mapping the inside surface of an article of footwear, comprising the steps of inserting the stereo vision camera of claim into the interior space of the footwear, activating a motor to bring the stereo camera into a predetermined position in the interior space of the footwear, taking stereo pairs of images and stored them within the memory, repeating the process to generating further stereo pairs of images for additional positions of the stereo vision camera inside the interior space, and creating a three dimensional model of the inside of the article of footwear.
